(19) United States
(12) Patent Application Publication
(10) Pub. No.: US 2011/0212045 A1
(43) Pub. Date: Sep. 1, 2011

(54) Cosmetic Compositions Containing An Ion Exchange Polymer, And Methods of Using The Same

(76) Inventors: Vasile Ionita-Manzatu, Old Bethpage, NY (US); Thomas Mammone, Farmingdale, NY (US); Lavinia C. Popescu, Jackson Heights, NY (US); Geoffrey Hawkins, Yardley, PA (US); Barbara Anne Wolf, Scarsdale, NY (US); Jack Lombardi, Massapoqua, NY (US)

(21) Appl. No.: 12/872,430

(22) Filed: Aug. 31, 2010

Related U.S. Application Data
(60) Provisional application No. 61/238,783, filed on Sep. 1, 2009.

(51) Int. Cl. A61K 8/81; A61K 8/73; A61Q 19/00; A61Q 19/08

(52) U.S. Cl. 424/78.03; 514/59

(57) ABSTRACT

Topical compositions for reducing the appearance of fine lines and wrinkles on the skin contain one or more ion exchange polymers, in a cosmetically or pharmaceutically acceptable carrier.

This application claims priority from U.S. 61/238,783 filed Sep. 1, 2009.

### FIELD OF THE INVENTION

The present invention relates to cosmetic compositions for topical application to improve the appearance of human skin. More specifically, the present invention relates to cosmetic compositions with skin-tightening and wrinkle-reduction functions as well as other skin benefits.

### BACKGROUND OF THE INVENTION

Skin is subject to damages by many extrinsic and intrinsic factors. Extrinsic factors include ultraviolet radiation (e.g., from sun exposure), environmental pollution, wind, heat, low humidity, gravity, harsh chemicals, abrasives, certain medicines (such as diuretics and retinoic acid), and the like. Intrinsic factors include chronological aging and other biochemical changes occurring within the skin. These extrinsic and intrinsic factors may result in visible signs of skin aging, such as the appearance of deep wrinkles and fine lines on the skin. For many people, wrinkles and fine lines are a reminder of the disappearance of their youth. Therefore, there is an enormous demand for cosmetic formulations that can effectively tighten the skin and reduce the appearance of wrinkles and/or fine lines.

Some cosmetic compositions comprise substances of natural origin, such as plant, egg, milk, or animal derivatives, as skin-tightening or wrinkle-reducing agents. For example, serum albumin exhibits a significant skin-tightening effect and has been used to reduce the appearance of wrinkles. However, serum albumin is uncomfortable to use, and it also leaves an unsightly while film on the skin.

Other cosmetic compositions have employed synthetic polymers as the skin-tightening or wrinkle-reducing agents. For example, U.S. Pat. No. 4,777,041 describes a wrinkle treatment formulation containing a gelable hydrophilic polyurethane and a precipitated silica thickener gelling agent that fills wrinkles when dried. However, the polyurethane and silica components of this formulation have various undesirable properties that make it unsuitable for widespread use.

Consequently, there is still a continuing need for improved cosmetic compositions that provide a suitable wrinkle reduction effect and that can be used to improve the appearance of the skin, without the drawbacks of the above-described conventional cosmetic compositions or formulations.

### SUMMARY OF THE INVENTION

The invention is directed to topical cosmetic compositions for reducing the appearance of fine lines and wrinkles on the skin, comprising one or more ion exchange systems in a cosmetically or pharmaceutically acceptable carrier.

The invention is further directed to a method of reducing the appearance of fine lines and wrinkles on the skin, comprising applying to saggy or wrinkled skin, a topical cosmetic composition as described herein. Preferably, but not necessarily, the topical composition is chronically applied to the lined or wrinkled or saggy skin for a period of at least one month, and at a frequency ranging from about once per week to about five times per day.

Other aspects and objectives of the present invention will become more apparent from the ensuring description, examples, and claims.

### DETAILED DESCRIPTION

### I. Definitions

Throughout this specification, the term “comprises” means that an element or a group of elements is not limited to those explicitly recited, but may or may not include additional elements.

### II. Ion Exchange Systems

It has been discovered that a topical cosmetic composition comprising one or more ion exchange systems has a surprising and unexpected effect in reducing appearance of fine lines and wrinkles on the skin. Within minutes of application of this topical cosmetic composition to saggy or wrinkled skin, the skin appears significantly tighter and the fine lines and wrinkles are significantly less visible.

Anion exchange systems include, for example, an insoluble or weakly soluble substrate that comprises tightly bound positively charged groups and loosely bound negative ions. Cation exchange systems include, for example, an insoluble or weakly soluble substrate that comprises tightly bound negatively charged groups and loosely bound positive ions. When the insoluble ion exchange system is placed in a solution that does not restrict the flow of ionic species, the loosely bound ions, generically referred to as “counter ions”, are able to be exchanged for ions of the same charge, taken up from the solution. Commonly used counter ions include chloride (Cl⁻) and sodium (Na⁺) ions.

Within ion exchange technology, various materials have been used for the matrix itself, such as aluminum silicates, synthetic resin, and polymers such as polysaccharides. A number of ion exchange systems that are suitable for cosmetic or pharmaceutical applications, may find use in the present invention, depending on the chemical composition and intended use of the final product. However, for topical applications, the ion exchange substrate is preferably polymeric and insoluble in water. In this case, a preferred polymeric substrate has the form of a crosslinked polymeric matrix. Such a matrix may be fashioned from one or more materials, such as polystyrene, polyacrylate, polymethacrylate and the like. These can be crosslinked with a cross-linking agent, such as divinylbenzene, for example. The crosslinked polymeric matrix may also be trade of a naturally occurring polysaccharide, such as dextrin, pullulan, and the like. Crosslinked polymers are preferred because in this application, they are more stable than non-crosslinked polymers.

Regarding the tightly bound charged group, phenolic hydroxyl groups, carboxyl groups, phosphonic acid groups and sulphonic acid groups are known to be used in cationic exchange systems, among which sulfonic acid group (which is a strongly acidic group) is most preferred. Aliphatic and aromatic amino groups such as tertiary amino groups and quaternary ammonium groups, pyridyl groups, imidazole groups, quaternary pyridinium groups, quaternary imidazolium groups, trimethylammonium groups, polyethylene amine groups, and the like, are known to be used in anionic exchange systems, among which quaternary ammonium groups and quaternary pyridinium groups (which are strongly basic groups) are most preferred.

The specifics of the tightly bound charged group determines several properties of the ion exchange system. These include: the type (cationic vs. anionic), the ionic strength (the degree to which loosely bound counter ions compete for binding sites on the ion exchange substrate), and the ion-exchange capacity (the number of charged groups per gram of dry ion exchanger). In general, a higher exchange capacity is achieved with weaker counter ions. A number of other factors also affect the exchange process, including temperature and pH of the solution, and porosity of the substrate, the degree of crosslinking of the substrate (greater degree of crosslinking generally means a lower capacity), and any other factors that might influence access to binding sites on the substrate.

In the present invention, as we will see, to optimize wrinkle reduction effect, it is desirable to use one or more ion exchange polymeric systems with sufficiently large ion exchange capacity (i.e. sufficiently weak ionic strength). For example, in the present invention, commercially viable compositions comprising efficacious ion exchange systems may have an ion-exchange capacity of 0.1 to 6.0 mmol/g of dry polymer. Furthermore, 0.3 to 4.0 mmol/g of dry polymer, may be more preferred. The water content of the ion-exchange polymer is preferably 0 to 90% by mass, more preferably 0.1 to 50% by mass, relative to the dry mass of the ion exchange polymer.

Particularly preferred anion exchange polymers for practice of the present invention include a high capacity, weak base, anion exchange resin, such as a polyacrylic matrix crosslinked with divinylbenzene, that supports complex amine functional groups. Such a resin is commercially available as Purolite® A830 from the Purolite Company (Bala Cynwyd, Pa.). Alternatively, a weak base anion exchange resin, such as a cross-linked dextran matrix that supports diethylaminoethyl functional groups is also particularly preferred. Such a resin is commercially available as DEAE Sephadex™ A-25 from GE Healthcare (Waukesha, Wis.).

Particularly preferred cation exchange polymers for practice of the present invention include a high capacity, weak acid cation exchange resin, such as a polymethacrylic matrix crosslinked with divinylbenzene, that supports carboxylic acid functional groups. Such a resin is commercially available as Purolite® C115 from the Purolite Company (Bala Cynwyd, Pa.). Alternatively, a weak acid cation exchange resin, such as a cross-linked dextran matrix that supports carboxymethyl functional groups, is also particularly referred. Such a resin is commercially available as CM Sephadex™ C-25 from GE Healthcare (Waukesha, Wis.).

The content of the ion exchange polymer in the topical cosmetic composition of the present invention is preferably 1 to 99% by total weight of the final composition, more preferably 10 to 90%. The ion exchange polymer, being water-insoluble, may be provided as micronized particles or microspheres, either hollow or solid, with a sufficiently small particle size that can be easily dispersed in a cosmetically or pharmaceutically acceptable carrier. The coverage and feel of the cosmetic on the skin may also inform the size of the particle. Too large a particle size may cause a negative sensorial experience for the user. Preferably, the ion exchange polymeric particles or microspheres are characterized by a largest dimension that has an average size ranging from about 1 to 150 μm, preferably from 1 to 100 μm, more preferably from 5 to 50 μm. For example, if the particles are spherical or roughly spherical, then the largest particle dimension will be the diameter. If the particles are platy, then the largest dimension will be the length of the long axis. Also, if the distribution of the largest dimension of the particles is multi-modal, then preferably, the peak of each mode will fall within 1 to 150 μm, preferably from 1 to 100 μm, more preferably from 5 to 50 μm.

### III. Theory of Operation

In a copending application (US2010-0030058), herein incorporated by reference, in its entirety), it was demonstrated that skin wrinkles associated with ageing can be characterized by an endogenous wrinkle electric field. Moreover, the course of wrinkle progression can be characterized by changes in the wrinkle electric field. Thus, '058 disclosed a new kind of wrinkle diagnosis, associated with the endogenous wrinkle electric field. Also, a new kind of wrinkle treatment was disclosed; treatments that affect a wrinkle by directly manipulating the wrinkle electric field. The results of any wrinkle treatment, it was disclosed, will manifest as measurable changes in the endogenous wrinkle electric field. A main object of the '058 invention is to treat wrinkles by applying to a wrinkle, a treatment that tends to reverse the electric field polarity of the wrinkle However, the '058 reference did not disclose topical wrinkle treatment products according to the present invention.

Without wishing to be bound by any one theory, in the wrinkle reduction compositions of the present invention there may be at least two related, but distinct mechanism of action at work. First, when applied to the surface of human skin, particularly wrinkled skin, compositions of the present invention seem to generate a static electrical charge on the skin surface, and an electric potential that extends deeper into the skin. As the '058 reference suggests, that electric potential can be formulated to manipulate the endogenous electric field of the wrinkle

Second, and apart from anything disclosed in the '058 reference, by depositing an ion exchange system on the skin surface, the concentration of loosely bound ions is artificially elevated in the upper layers of the epidermis (or we could say that the concentration of pure water is reduced in the upper layers of the epidermis). To the extent that the layers of the epidermis act as a semi-permeable membrane, the result is an osmotic imbalance. To the extent that the layers of the epidermis act as a semi-permeable membrane, the layers of the epidermis will allow the passage of water, but not the passage of ions of the ion exchange system. Osmotic balance is restored when water molecules migrate from the deeper layers of the epidermis to the more superficial layers. The increase in water content of the more superficial layers of the epidermis manifests visually, as a decrease in the number and/or severity of visible lines and wrinkles in the skin, and as a skin tightening effect. Immediately or within several minutes after topical application, the skin is visibly less wrinkled. The effect may persist from one to several hours, and reapplication can be used to extend the effect.

The wrinkle reduction effect is expected to be greater when the topical composition creates a greater imbalance is osmotic pressure. A greater imbalance in osmotic pressure is achieved if the counter ions are only weakly held to the ion exchange matrix (when the ion exchange system has a greater ion exchange capacity). Thus, weaker ion exchange systems are preferred over stronger. Also, to the extent that osmotic pressure is a colligative property, it does not matter is the counter ions are positively or negatively charged. Either way, water is expected to flow from deeper layers, were pure water concentration is higher, to more superficial layers, where pure water concentration has been reduced by the counter ions. However, we also hypothesize that the electric field created by the counter ions causes a change in the permeability of the epidermis, making the epidermis more permeable to the flow of water. If so, then the electric charge on the counter ions may be relevant to the overall process of osmosis, as herein described.

### IV. Optional Ingredients

Compositions of the present invention may contain a variety of optional ingredients including but not limited to those set forth herein. In general, any particular ingredient or combination of ingredients may be used in compositions of the present invention, with the proviso that the ingredients do not completely negate the skin's response to the exchange ions applied to the skin.

Regarding water in the composition, the compositions may be emulsions, gels, solutions, suspensions, powders or anhydrous. Water-in-oil and/or oil-in-water emulsions may be suitable, and may include from about 0.01-99%, preferably from about 0.5-95%, more preferably from about 1-90% by weight of the total composition of water and from about 0.01-98%, preferably from about 0.1-95%, more preferably from about 0.5-90% by weight of the total composition of oil. If present in the solution or gel form, the composition may comprise from about 0.01-99% by weight of the total composition of water and other optional ingredients. If present in the powder or anhydrous form, the composition may be substantially free of water, i.e., with water content of less than 5% by weight, and more preferably less than 1% by weight of the total composition. If it is desirable to maximize the effect of osmotic pressure as described above, then it may be preferable to keep the amount of free water in the composition low, for example, from 0-50%; more preferably, from about 0-20%; even more preferably about 0-10%, and most preferably about 0-5%, by weight of the total composition. In this sense, anhydrous compositions may sometimes be preferred.

The topical cosmetic composition of the present invention may further contain one or more skin care active ingredients or skin care actives. The term “skin care active ingredients” or “skin care actives” as used herein refers to agents that provide benefits to the skin rather than merely improving the physical characteristics of the topical composition. For example, the topical composition may comprise anti-aging agents that are capable of protecting the skin against photo- or chrono-aging by scavenging free radicals, preventing lipid peroxidation, inactivating lipogenase, inhibiting undesired enzymatic activities, and stimulating collagen synthesis. The topical composition may also include anti-acne agents, enzyme-inhibiting agents, collagen-stimulating agents, sunscreen agents, antioxidants, exfoliants, agents for the eradication of age spots, keratoses and wrinkles, analgesics, anesthetics, antibacterials, antiyeast agents, antifungal agents, antiviral agents, antidandruff agents, antidermatitis agents, antipruritic agents, antiemetics, anti-inflammatory agents, antihyperkeratolytic agents, antiperspirants, antipsoriatic agents, antiseborrheic agents, antiwrinkle agents, antihistamine agents, skin lightening agents, depigmenting agents, vitamins, corticosteroids, self-tanning agents, hormones, retinoids such as retinoic acid and retinol, topical cardiovascular agents, clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, hydroquinone, minocycline, naproxen, ibuprofen, theophylline, cromolyn, albuterol, topical steroids such as hydrocortisone, hydrocortisone 21-acetate, hydrocortisone 17-valerate, and hydrocortisone 17-butyrate, betamethasone valerate, betamethasone diproprionate, benzoyl peroxide, crotamiton, propranolol, promethazine, vitamin A palmitate, vitamin E acetate and mixtures thereof.

The topical cosmetic composition of the present application further comprises a cosmetically acceptable vehicle. For purpose of the present invention, cosmetically acceptable vehicles are substances that can be used to formulate the above-described active ingredients into a cream, gel, emulsion, liquid, suspension, oil, lotion, paste, or powder that can be topically applied. Substances which may be formulated into the topical composition of the present application include, but are not limited to: moisturizing agents, astringent agents, chelating agents, surfactants, emollients, preservatives, stabilizers, thickeners, humectants, pigments, etc. Preferably, but not necessarily, such vehicles aid the formation of a protective layer on the skin.

The vehicle or vehicles can present in the topical composition of the present invention at an amount ranging from about 1% to about 99.9%, preferably from about 50% to about 99.5%, more preferably from about 70% to about 99%, and most preferably from about 80% to 90% by total weight of the topical composition.

Optional ingredients that can be used for formulating the topical cosmetic compositions of the present invention are described in U.S. 61/238,783 which is herein incorporated by reference, in its entirety.

### V. Forms of the Composition

The types of compositions useful for the present invention include moisturizer, serum, lotion, toner, cream, gel and the like, for imparting the skin with a smoother, more youthful appearance. The compositions may also include color cosmetic compositions such as blush, eyeshadow, lipstick, lip gloss, foundation, concealer, and the like, for enhancing the appearance of the skin. The compositions may be in the form of skin creams or lotions, toners, gels, blush, eyeshadow, foundation, concealer, and so on. Preferred are color cosmetic compositions such as blush, eyeshadow, foundation, concealer, and the like.

Suitable foundation makeup, concealer, blush, or eyeshadow compositions are generally in a powdery anhydrous form or liquid emulsion form, and have water, oil, pigments and, in the case where the compositions are in the emulsion form, optionally at least one surfactant, in the percentage ranges as set forth herein.

Suitable lip treatment compositions include lipstick, lip gloss, lip balm and the like. Such compositions may be in the aqueous emulsion form, containing water, oil, and pigments in the amounts set forth herein. More often lip treatment compositions will be in the anhydrous form, containing pigments, oils, and oil phase structuring agents in the amounts set forth herein.

Skin creams and lotions are preferably in the emulsion form, containing ranges of water and oil as set forth herein. When the ion exchange systems as described herein are incorporated into such compositions, the compositions exhibit improved effectiveness in reducing the appearance of skin wrinkles, lines, or depressions and otherwise cosmetically improving the appearance of skin.

### VI. Methods

The invention also includes a method for improving the appearance of aging skin, particularly reducing the appearance of fine lines and wrinkles on the skin, by applying a topical cosmetic composition comprising an ion exchange polymer in a cosmetically or pharmaceutically acceptable carrier to the skin that contains such fine lines and wrinkles. Such topical compositions can be applied to eyes, chin, neck and other facial areas to reduce sagginess of the skin and reduce the appearance of fine lines and wrinkles. It can also be applied to other areas of the body that contain saggy or wrinkled skin. Compositions of the invention provide immediate, visible skin-tightening and wrinkle-reduction effects. They are effective in reducing both fine lines and deeper, greatly visible wrinkles. Further, compositions of the invention may be left on the skin for a relatively long period of time to provide long-term skin benefits. This may be due to the static nature of the exchange ions once they are positioned on the skin.

The topical cosmetic compositions of the present invention may also be used for treating or regulating other skin disorders associated with insufficient water in the epidermis. The topical cosmetic compositions of the present invention may also be used for treating or regulating other types of skin disorders by the addition of one or more actives effective for preventing, retarding, arresting, or treating such other skin disorders. For example, the topical cosmetic compositions of the present invention may be used for regulating dry skin, xerosis, dandruff, keratoses, psoriasis, eczema, age spots, lentigines, melasmas, blemished skin, hyperpigmentation, hyperkeratosis, inflammatory dermatoses, rosacea, and age-related skin changes.

The methods of application in the present invention will depend on the ultimate intended use of composition. The topical cosmetic composition can be applied locally to the saggy or wrinkled skin, or it can be applied to the entire body of the user. The topical cosmetic composition of the present invention may be applied to the skin on an as-needed basis, to achieve immediate wrinkle reduction and moisturization results (typically observable within several minutes). Alternatively, the topical cosmetic composition can be applied to the skin repeatedly according to a pre-set schedule. The amount of the topical cosmetic composition applied each time, the area of application, the duration of application, and the frequency of application can vary widely, depending on the specific need of the user. For example, the topical cosmetic composition can be applied for a period of at least one month and at a frequency ranging from about once per week to about five times per day. For another example, the topical cosmetic composition is applied for a period of about six months and at a frequency ranging from about three times a week to about three times per day, and preferably about once or twice per day.

The topical cosmetic composition of the present invention may be applied directly to clean skin, before application of any moisturizer, foundation, make-up, etc. Alternatively, the topical cosmetic composition of the present invention can be applied over moisturizer, and optionally over foundation and/or make-up. When used in conjunction with other topical compositions, care should be taken to ensure that the other topical composition does not interfere with the skin's response to the exchange ions applied to the skin.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

### VII. Examples

The following examples of compositions according to the present invention are illustrative only.

### Example 1

### Neutral Charge Cream Base

| | |
|---|---|
| Deionized water | 97.0% |
| Urea Crystale Extra Pure usp | 0.5% |
| Aristoflex AVC | 0.5% |
| BDP-500 | 1.0% |
| Phenoxetol | 1.0% |

### Example 2

### Positively Charged Cream

| | |
|---|---|
| Deionized water | 96.0% |
| Urea Crystale Extra Pure usp | 0.5% |
| Aristoflex AVC | 0.5% |
| BDP-500 | 1.0% |
| Phenoxetol | 1.0% |
| DEAE Sephadex | 1.0% |

DEAE Sephadex is a weakly basic anion exchanger having diethylaminoethyl as its functional group.

### Example 3

### Negatively Charged Cream

| | |
|---|---|
| Deionized water | 96.0% |
| Urea Crystale Extra Pure usp | 0.5% |
| Aristoflex AVC | 0.5% |
| BDP-500 | 1.0% |
| Phenoxetol | 1.0% |
| CM Sephadex | 1.0% |

CM Sephadex is a weakly acidic cation exchanger having carboxymethyl as its functional group.

### Example 4

### Neutral Charged Cream Base

| | |
|---|---|
| Deionized water | 93.25% |
| Aristoflex AVC | 0.85% |
| Keltrol CG | 0.2% |
| BDP-500 | 1.0% |
| Hydrolyte | 4.0% |
| Phenoxetol | 0.7% |

### Example 5

### Negatively Charged Cream

| | |
|---|---|
| Deionized water | 91.25% |
| Aristoflex AVC | 0.85% |
| Keltrol CG | 0.2% |
| BDP-500 | 1.0% |
| Hydrolyte | 4.0% |
| Phenoxetol | 0.7% |
| Purolite A830MR | 2.0% |

Purolite A830MR is a weakly basic, polystyrene, anion type exchanger, which has as functional ion a free base

### Example 6

### Positively Charged Cream

| | |
|---|---|
| Deionized water | 91.25% |
| Aristoflex AVC | 0.85% |
| Keltrol CG | 0.2% |
| BDP-500 | 1.0% |
| Hydrolyte | 4.0% |
| Phenoxetol | 0.7% |
| Purolite C115HMR | 2.0% |

Purolite C115HMR is weakly acidic, polystyrene, cation type exchanger, which has H⁺ as its functional ion.

### Example 7

### Electrical Polarity Evaluation

In order to evaluate the electrical polarity of the compositions of examples 1-6, a voltaic element was created as follows. A 30 g sample of the composition to be measured and 30 g of distilled water were put into separate glass beakers. The test sample and the distilled water were electrically connected with a Ni—Cr ion steel wire, about 1 mm diameter, 2 inches length, and having 0.01-5 ohms resistance. The test sample and distilled water serve as the electrodes of the voltaic element.

The electrical potential and polarity of this voltaic element were measured in a way that is similar to measuring the potential and polarity of a conventional battery, i.e. by connecting a voltmeter to the electrodes of the voltaic element (digital multimeter model GDM 8034, manufactured by Instek Corporation USA). In order not to contaminate the sample, the electrodes of the voltmeter were connected to the to the test sample and distilled water through platinum wires, about 0.5 mm. diameter and a resistance of about 0.01-1 ohms. The results of these measurements were as follows.

| | Electrical | |
|---|---|---|
| Test Sample | potential (mV) | Electrical Polarity |
| Neutral Charge Cream (Ex 1) | +0.6 | effectively neutral |
| Positively Charged Cream (Ex 2) | +208 | strong positive |
| Negatively Charged Cream (Ex 3) | −89 | negatively charged |
| Neutral Charged Cream (Ex4) | +35 | slightly positive |
| Negatively Charged Cream (Ex 5) | −340 | strong negative |
| Positively Charged Cream (Ex 6) | +330 | strong positive |

Thus, the compositions of the present invention, when applied to the skin, are able to generate a static electrical potential that penetrates the skin and modulates the natural skin electric potential. This applied electric potential may be used to modulate the electric field of skin wrinkles for an expected benefit. And/or compositions of the present invention, when applied to the skin, are able to effect osmosis of water from the lower layers of the skin into higher layer of the skin, possibly altering the permeability of the epidermis as regards osmosis. At any rate, the effects observed include wrinkle reduction and skin tightening.

What is claimed is:

1. A topical cosmetic composition for reducing the appearance of fine lines and wrinkles on the skin comprising one or more ion exchange polymers in a cosmetically or pharmaceutically acceptable carrier.

2. The composition of claim 1 wherein at least one of the ion exchange polymers is an anion exchange polymer.

3. The composition of claim 2 wherein the anion exchange polymer is a weak base anion exchange polymer containing a polyacrylic matrix crosslinked with divinylbenzene and supporting amine functional groups.

4. The composition of claim 2 wherein the anion exchange polymer is a weak base anion exchange polymer containing a crosslinked dextran matrix supporting diethylaminoethyl functional groups.

5. The composition of claim 1 wherein at least one of the ion exchange polymers is a cation exchange polymer.

6. The composition of claim 5 wherein the cation exchange polymer is a weak acid cation exchange polymer containing a polymethylacrylic matrix crosslinked with divinylbenzene and supporting carboxylic acid function groups.

7. The composition of claim 5 wherein the cation exchange polymer is a weak acid cation exchange polymer containing a crosslinked dextran matrix supporting carboxymethyl functional groups.

8. A method for reducing the appearance of fine lines and wrinkles on the skin, comprising applying to wrinkled skin a topical cosmetic composition comprising one or more ion exchange polymers in a cosmetically or pharmaceutically acceptable carrier.

9. The method of claim 8 wherein at least one of the ion exchange polymers is an anion exchange polymer.

10. The method of claim 9 wherein the anion exchange polymer is a weak base anion exchange polymer containing a polyacrylic matrix crosslinked with divinylbenzene and supporting amine functional groups.

11. The method of claim 9 wherein the anion exchange polymer is a weak base anion exchange polymer containing a crosslinked dextran matrix supporting diethylaminoethyl functional groups.

12. The method of claim 8 wherein at least one of the ion exchange polymers is a cation exchange polymer.

13. The method of claim 12 wherein the cation exchange polymer is a weak acid cation exchange polymer containing a polymethylacrylic matrix crosslinked with divinylbenzene and supporting carboxylic acid function groups.

14. The method of claim 12 wherein the cation exchange polymer is a weak acid cation exchange polymer containing a crosslinked dextran matrix supporting carboxymethyl functional groups.
